(19) United States
(12) Patent Application Publication
(10) Pub. No.: US 2008/0081055 A1
(43) Pub. Date: Apr. 3, 2008

(54) FILM WITH IMPROVED DISSOLUTION, COSMETIC PRODUCT

(75) Inventor: Guillaume CASSIN, Villebon Sur Yvette (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/834,796

(22) Filed: Aug. 7, 2007

Related U.S. Application Data
(60) Provisional application No. 60/839,119, filed on Aug. 22, 2006.

(30) Foreign Application Priority Data
Aug. 10, 2006 (FR) 06 53343

(51) Int. Cl. A61K 8/89; A61K 8/02; A61Q 99/00; C08L 83/04; A61K 8/73

(52) U.S. Cl. 424/401; 424/78.03; 524/27; 524/35; 524/55

(57) ABSTRACT

Water-soluble anhydrous film containing (i) at least one water-soluble or water-dispersible film-forming polymer, (ii) at least one polysaccharide thickening agent, (iii) at least one water-soluble or water-dispersible oxyalkylenated polysiloxane and (iv) one or more plasticizers chosen from polyols. The invention also relates to the product obtained by mixing at least one film with an aqueous composition and to a kit for the formulation of a cosmetic product.

### REFERENCE TO PRIOR APPLICATIONS

This application claims priority to U.S. provisional application 60/839,119 filed Aug. 22, 2006, and to French patent application 0653343 filed Aug. 10, 2006, both incorporated herein by reference.

### FIELD OF THE INVENTION

The present invention relates to water-soluble anhydrous films and to their uses in the cosmetic or dermatological field, in particular in dissolution in a composition appropriate for topical application. The invention also relates to a kit comprising the film and to an aqueous composition in which this film is dissolved.

The invention also relates to the use of the mixture obtained from the film and from the composition in the treatment of the skin, mucous membranes, superficial body growths or hair.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BRIEF DESCRIPTION OF THE DRAWINGS

FIG. 1 shows the test arrangement used in the dissolution test of the film of Example 1 in comparison with that of the comparative example.

FIG. 2 shows the curves obtained with the films corresponding to the examples according to the invention (Example 1) and the comparative example.

### BACKGROUND OF THE INVENTION

Apart from the usual cosmetic products which are provided in the liquid, gel or solid form, it is known to use cosmetic preparations in the form of thin water-soluble films, as described, for example, in the document JP-A-2002/212027 or in the documents US-A-2004/0071755, WO-A-2002/05789, US-A-2002/0127254 and WO-A-2003/075812, which describe the preparation of anhydrous polymeric films for direct administration of cosmetic compositions to prewetted skin. Furthermore, the document US-A-2003/0186826 describes a dry cosmetic composition based on polymers and on surfactants to be administered to the skin or hair with water.

Furthermore, the document FR-A-2 868 949 describes kits comprising one or more thin anhydrous films and a third composition into which this or these films are introduced. At the moment of application, the thin anhydrous film or films are dissolved at the time of use with the fluid composition to form a new composition to be applied to the skin, mucous membranes or superficial body growths. Such a cosmetic composition makes it possible to solve the problems of colloidal or chemical stability, to adjust the final rheology of the preparation and its concentration of active principles or also to provide formulations “on request”. In addition, this makes it possible to combine, in the same composition, molecules which are incompatible with one another, which is a more economical solution than the application of two successive compositions. Furthermore, this makes it possible to be able to deposit, on the skin, doses of active principles which are significantly higher than with a conventional formulation.

In addition, the document FR-A-2 871 685 describes a shaving kit comprising an anhydrous film soluble in a solvent, such as water.

However, these anhydrous films exhibit the disadvantage of having imperfect dissolution properties, that is to say that the dissolution of these films is not complete within a reasonable time (the term “reasonable time” is understood here to mean a time of less than 30 seconds). In point of fact, in order to have a satisfactory final composition, it is necessary for the dissolution of the film to be complete within a reasonable time in order for the user not to have to wait in order to apply the product. In addition, the incomplete dissolution of the film is effected by the appearance of particularly unsightly clumps during the dissolution of the film and during its application to the skin or to any other keratinous substance.

### SUMMARY OF THE INVENTION

The need thus remains to have available anhydrous films having kinetics of dissolution in fluid formulations which are fast and which allow optimized use of such films. The inventor has found, surprisingly, that the addition of oxyalkylenated polysiloxanes to these thin films makes it possible to achieve the desired goal.

### DETAILED DESCRIPTION OF THE PREFERRED EMBODIMENTS

A subject-matter of the present invention is thus a water-soluble anhydrous film comprising (i) at least one water-soluble or water-dispersible film-forming polymer, (ii) at least one polysaccharide thickening agent, (iii) at least one water-soluble or water-dispersible oxyalkylenated polysiloxane and (iv) at least one plasticizer chosen from polyols.

Another subject-matter of the invention is the product obtained by mixing, at the time of use, at least one film as defined above with a suitable amount of an aqueous composition.

Another subject-matter of the invention is a kit for the formulation of a cosmetic product comprising:
i) an aqueous composition;
ii) at least one water-soluble anhydrous film as defined above.

A cosmetic product is obtained by adding one or more films to the aqueous composition. The films added can have an identical or different composition.

A further subject-matter of the invention is a kit for the customized formulation of a product, preferably a cosmetic product, in particular a care or makeup product, comprising:
i) an aqueous composition;
ii) a plurality of identical or different water-soluble anhydrous films intended to be mixed with the aqueous composition in order to form the cosmetic product; and
iii) optionally instructions, in particular on directions for use,
   in order to formulate the product in tailor-made fashion according to the number of identical or different water-soluble anhydrous films to be mixed with the composition.

The term “customized formulation” is understood to mean a formulation suited to the need of the consumer at the moment of use.

The final product is obtained by mixing, at the time of use, one or more water-soluble anhydrous films and an appropriate amount of the aqueous composition. The term “appropriate amount” of the aqueous composition is understood to mean an amount such that the film or films rapidly dissolve therein. This amount can range, for example, from 10 to 1000 mg, preferably from 50 to 800 mg and better still from 100 to 500 mg. The appropriate dose of aqueous composition can be obtained by using single-dose presentation forms, such as sachets, tubes, vials, prefilled syringes, soft capsules, or shells or trays made of thermoformed plastic. An appropriate dose can also be obtained from a multidose presentation using a system distributing a predefined dose. Such a system can be a pump-dispenser bottle, an aerosol, a pipette, a graduated syringe or a dropper.

Yet other subject-matters will become apparent in the further description which follows.

A. Water-Soluble Anhydrous Films

The term “film” is understood to mean, in the present patent application, a thin solid. The term “thin” is understood to mean a solid having a thickness of at most 1000 μm.

This film can be grasped, that is to say that it generally has a dimension appropriate for being able to be easily handled by the user. It can have a square, rectangular or disc shape or any other shape. Each film generally has a thickness of 10 μm to 1000 μm, preferably of 20 to 500 μm and better still 50 to 300 μm. It can have a surface area of 0.25 to 25 cm² and preferably of 2 to 10 cm².

Furthermore, the term “anhydrous film” is understood to mean, in the present patent application, a film comprising less than 15′ by weight of water (0 to 150 by weight of water), preferably less than 10% by weight (0 to 10′ by weight of water) and better still less than 50 by weight (0 to 5% by weight of water), with respect to the total weight of the film, and more preferably not comprising water.

In addition, the term “water-soluble film” is understood to mean, in the present patent application, a film which dissolves in water.

The film which is used for cosmetic or dermatological purposes comprises a physiologically acceptable medium, that is to say a medium compatible with the skin, mucous membranes, hair and scalp.

Oxyalkylenated Polysiloxanes

The oxyalkylenated polysiloxanes used according to the invention are water-soluble or water-dispersible. The term “water-soluble or water-dispersible” is understood to mean polysiloxanes having a solubility in water, measured at 25° C., at least equal to 0.1 gram/litre (g/l) (production of a macroscopically isotropic and transparent solution which may or may not be coloured). This solubility is preferably greater than or equal to 1 g/l.

These polysiloxanes are preferably chosen from water-soluble silicones comprising at least one terminal or pendent monovalent polyoxyalkylene group which, introduced at 0.05% by weight into an aqueous solution, are capable of reducing the surface tension of the water to a value of less than 35 mN/m and preferably of less than 30 mN/m.

The polysiloxanes can in particular be polydimethylsiloxanes or PDMSs.

The oxyalkylenated polysiloxanes in accordance with the invention are more preferably chosen from the water-soluble silicones of the following general formula (a):
R²₃SiO(R²₂SiO)ₚ(R²PESiO)_{q}SiR²₃ (a)
in which:

the R² radicals, which are identical or different, denote a monovalent hydrocarbon radical chosen from alkyl, aryl and aralkyl radicals having at most 10 carbon atoms;

p varies from 0 to 150, preferably from 0 to 100 and more preferably from 0 to 30;

q varies from 1 to 12, preferably from 1 to 10 and more preferably from 1 to 8;

the polyether group PE has the following formula (b):
—CₓH₂ₓ(OC₂H₄)_{y}(OC₃H₆)_{z}OR³ (b)
in which:

x varies from 1 to 8 and preferably varies from 2 to 4 and is more preferably equal to 3;

y is greater than 0;

z is greater than or equal to 0; the values of y and z being such that the total molecular weight of the polyoxyalkylene portion of the polyether group PE varies from 200 to 10 000 and more preferably from 350 to 4000;

R³ denotes hydrogen, a C₁-C₈ alkyl group or a C₂-C₈ acyl group.

It should be noted that, when z is other than 0, the polyoxyethylene and polyoxypropylene units can be distributed randomly along the polyether chain PE or distributed in blocks or else distributed both in blocks and randomly.

Preferably, the R² radicals are chosen from C₁-C₄ alkyl groups and hexyl, phenyl and benzyl groups. More particularly, the R² radicals are chosen from alkyl groups, such as methyl, ethyl or butyl groups. More particularly still, they denote the methyl radical.

Preferably, the R³ radicals are chosen from C₁-C₄ alkyl groups and more particularly still denote the methyl radical.

The water-soluble silicones of formula (a) can be obtained according to the process described in the document U.S. Pat. No. 4,847,398.

Use is preferably made, among the water-soluble silicones of formula (a), of those of following formula (a′):
Me₃SiO(MeSiO)ₚ(MePESiO)_{q}SiMe₃ (a′)
in which p and q have the same values as indicated above for the formula (a), Me denotes the methyl radical and PE denotes:
—(CH₂)₃(OC₂H₄)_{y}(OC₃H₆) OR³ (b′)
where y and z have the same values as indicated above for the formula (b) and R³ denotes hydrogen or a C₁-C₄ alkyl group and more particularly the methyl radical.

Mention may be made, as other family of water-soluble polysiloxanes which can be used according to the invention, of the branched silicones of following formula (c):
(MeSiO)_{q-2}[SiOMe2)_{p/q}OPE]_{q} (c)
where p and q have the same values as indicated above in the formula (a), Me signifies methyl and PE denotes the group of following formula (d):
—(OC₂H₄)_{y}(OC₃H₆)_{z}R³ (d)
where y and z have the same values as indicated above in the formula (b) and R³ denotes a C₁-C₄ alkyl group and more particularly the methyl radical.

It is possible, of course, to use a mixture of the silicones of formula (a) and of formula (c).

Such oxyalkylenated polysiloxanes are sold, for example, by OSI under the trade names Silwet L-7210® (INCI name: Dimethicone Copolyol), Silwet L-7220® (INCI name: Dimethicone Copolyol); Silwet L-7002®, Silwet L-7600® (INCI name: Dimethicone Copolyol), Silwet L-7604® (INCI name: PEG-8 Dimethicone), Silwet L-7605®, Silwet L-7607®, Silwet 1614, Silwet L-7657®, Silwet L-7200® (INCI name: Dimethicone Copolyol), Silwet L-7230®, Silsoft 305® (INCI name: Dimethicone Copolyol), Silsoft 820® (INCI name: Dimethicone Copolyol), Silsoft 880® (INCI name: PEG-12 Dimethicone), Tego Wet 260®, Tego Wet 500®, Tego Wet 505 and Tego Wet 510®.

Values of surface tensions at 25° C. of the aqueous solutions comprising 0.05% (by weight) of different oxyalkylenated polysiloxanes are collated in the following table:

| Oxyalkylenated | Surface tension at 0.05% in water |
|---|---|
| polysiloxane | (mN/m) |
| Tegowet 500 | 33 |
| Tegowet 510 | 29 |
| Silsoft 880 | 26 |
| Silsoft 305 | 21 |

The amount of oxyalkylenated polysiloxane(s) in the film according to the invention can range, for example, from 0.5 to 30% by weight, preferably from 1 to 20% by weight, with respect to the total weight of the film.

Film-Forming Polymers

The film comprises one or more water-soluble or water-dispersible film-forming polymers. The term “water-soluble or water-dispersible” is understood to mean polymers having a solubility in water, measured at 25° C., at least equal to 0.1 gram/litre (g/l) (preparation of a macroscopically isotropic and transparent solution which may or may not be coloured). This solubility is preferably greater than or equal to 1 g/l. Like the other compounds, these polymers had to be physiologically acceptable, that is to say compatible with the skin, mucous membranes, hair and scalp.

The term “film-forming polymer” is understood to mean a polymer capable of forming, by itself alone or in the presence of an additional agent which is able to form a film, a continuous film and preferably a film having a cohesion and mechanical properties such that the film can be isolated from a support.

These polymers are catalogued under the heading “Film Formers” in the cosmetic dictionary “International Cosmetic Ingredient Dictionary and Handbook” (see, for example, pages 2903 to 2906 of the ninth edition, 2002).

The film-forming polymers can be chosen, for example, from:
vinyl polymers, such as polyvinyl acetate, polyvinyl-pyrrolidones, copolymers of methyl vinyl ether and of maleic anhydride, the copolymer of vinyl acetate and of crotonic acid, copolymers of vinylpyrrolidone and of vinyl acetate, copolymers of vinylpyrrolidone and of caprolactam, or polyvinyl alcohols;
film-forming cellulose derivatives, such as hydroxyethylcellulose, hydroxypropylcellulose, hydroxypropylmethylcellulose, methylcellulose, ethylhydroxyethylcellulose, carboxymethylcellulose and quaternized cellulose derivatives;
starches and their derivatives;
optionally modified polymers of natural origin, such as pullulan, pectin, mannan, galactomannans, glucomannans and their derivatives, gum arabic, guar gum, xanthan gum, karaya gum; alginates, carrageenans, ulvans and other algal colloids; hyaluronic acid and its derivatives; shellac, sandarac gum, dammars, elemis, copals; deoxyribonucleic acid; mucopolysaccharides, such as hyaluronic acid, chondroitin sulphate;
anionic, cationic, amphoteric or nonionic polymers derived from chitin or from chitosan;
protein polymers, such as wheat or soybean protein; keratin and its derivatives, for example keratin hydrolysates and sulphonic keratins; casein; albumin; collagen; glutelin; glucagon; gluten; zein; gelatins and their derivatives;
acrylic copolymers of phosphorylcholine, such as poly[2-(methacryloyloxyethyl)phosphorylcholine], sold under the name Lipidure HM by NOF Corporation (INCI name: Polyphosphorylcholine glycol acrylate);
anion-cation complexes of gum arabic/gelatin or gum arabic/chitosan or collagen/glycosoaminoglycan type;
and the mixtures of these polymers.

According to a preferred embodiment of the invention, the film-forming polymer is chosen from vinyl polymers, cellulose derivatives and their mixtures.

Preferably, the vinyl polymer is polyvinyl acetate (PVA), which is prepared by radical polymerization of the vinyl acetate monomer, followed by hydrolysis. Use may be made in particular of polyvinyl acetate hydrolysed to 88%, such as that sold under the name Celvol 540 PV Alcohol by Celanese Chemicals.

Preferably, the cellulose derivatives are chosen from hydroxypropylcellulose (HPC) and hydroxypropylmethylcellulose (HPMC). These polymers are soluble in water and in organic solvents. This makes it possible to increase the range of solubility of the films comprising them. The choice of the molecular weight of these cellulose polymers has to be carefully made in order to enhance the dissolution of the films.

The HPCs preferably used are those sold by Hercules under the names:
Klucel® MF, the molecular weight of which is 850 000 (viscosity 4000-6500 mPa at 2% in water);
Klucel® EF, the molecular weight of which is 80 000 (viscosity 300-600 mPa at 10% in water).

The HPMC preferably used is hydroxypropylmethyl-cellulose with a viscosity of 40-60 cPs at 2% in water at 20° C., sold by Sigma-Aldrich.

Preferably, the film-forming polymer is chosen from polyvinyl acetate, hydroxypropylcellulose, hydroxypropylmethylcellulose and their mixtures.

The amount of water-soluble film-forming polymer(s) in the film according to the invention can range, for example, from 10 to 95% by weight, in particular from 20 to 70% by weight and more particularly from 30 to 60% by weight, with respect to the total weight of the film.

Use may be made, in the film of the invention, of a polymer which is both a film-forming polymer and a thickening polymer, for example chosen from cellulose derivatives and polymers of natural origin which can be both film-forming and thickening. The amount for use remains that indicated above: for example from 10 to 95% by weight, in particular from 20 to 70% by weight and more particularly from 30 to 60% by weight, with respect to the total weight of the film.

Polysaccharide Thickening Agent

The film according to the invention comprises at least one polysaccharide thickening agent.

The polysaccharide thickening agents used in the film according to the invention are chosen from polysaccharides possessing a gelling power. The term “gelling power” is defined as the fact that, at a concentration of greater than or equal to 0.5% by weight in water, the viscosity of the solutions thus obtained is greater than or equal to 0.01 Pa·s for a shear rate equal to 1 s⁻¹, the measurements being carried out at 25° C. using a RheoStress RS150 rheometer from Haake in cone/plate configuration, the measurements of the measuring cone being as follows: diameter of 60 mm and angle of 2°.

The polysaccharide thickening agents can be chosen in particular from gum arabic, gum ghatti, gum karaya, locust bean gum, guar gum, tamarin gum, xanthan gum, gellan, pectins, gum tragacanth, agar-agar, alginates, carrageenan, furcelleran, konjac gum and cellulose derivatives, and their mixtures.

According to a preferred embodiment of the invention, the polysaccharide thickening agents are chosen from carrageenans, which are linear polysaccharides extracted from certain red algae of the Rhodophyceae class. They are composed of alternating β-1,3 and α-1,4 galactose residues, of numerous galactose residues which can be sulphated. There exist three types of carrageenans, referred to as κ-carrageenan, ι-carrageenan and λ-carrageenan. This family of polysaccharides is described, for example, in Chapter 3 of the book “Food Gels”, edited by Peter Harris, Elsevier 1989.

Use may in particular be made of the carrageenan sold under the name Satiagum UTC 10 by Degussa.

The amount of thickening agent(s) in the film according to the invention can range, for example, from 0.5 to 40% by weight, in particular from 1 to 20% by weight and more particularly from 5 to 10% by weight, with respect to the total weight of the film.

Plasticizer

The film additionally comprises one or more plasticizers chosen from polyols, such as glycerol, sorbitol, mono- and/or disaccharides, dipropylene glycol, butylene glycol, pentylene glycol or polyethylene glycols, such as PEG-400. It can additionally comprise one or more plasticizers other than polyols. The amount of plasticizer(s) can range, for example, from 1 to 40% by weight and better still from 2 to 15% by weight, with respect to the total weight of the film.

The films can be packaged in an article which makes it easier to grasp them, such as that described in the document FR-A-2 863 167, the content of which is included in the present patent application by reference. The films can in particular be packaged in a dispensing plastic box, in an individual bag or in a blister pack. The films can be packaged in a case of the type with a drawer or with a lid articulated on a base, the case being able to comprise means intended to facilitate the dispensing of the articles. The dispensing means can be, for example, of the type of those described, for example, in the documents U.S. Pat. No. 2,973,882, GB-A-2 358 627, CH-A-461 025 or U.S. Pat. No. 6,578,732.

Aqueous Composition

The term “aqueous composition” is understood to mean, in the present patent application, a composition comprising at least water. The amount of water can vary according to the formulation of the composition but it is generally at least 20% by weight, with respect to the total weight of the composition.

The aqueous composition, apart from water, can comprise a water-soluble organic solvent chosen, for example, from lower monoalcohols comprising from 1 to 8 carbon atoms and in particular 1 to 6 carbon atoms, such as ethanol, isopropanol, propanol or butanol; polyethylene glycols having from 6 to 80 ethylene oxides; polyols, such as propylene glycol, isoprene glycol, butylene glycol, glycerol or sorbitol; acetone; and their mixtures.

It is a composition which is more or less fluid, in contrast to a solid composition. The term “composition which is more or less fluid” is understood to mean, in the present patent application, a composition for which the viscosity can be measured and ranges from the liquid to the semisolid (cream or soft paste). The viscosity can range, for example, from 1 to 20 000 mPa·s (1 to 20 000 cpoises), preferably from 1 to 15 000 mPa·s, this viscosity being measured at 25° C. using the Rheomat RM180 from Rheometric Scientific, this device being equipped with a different spindle according to the viscosities.

This composition preferably comprises a physiologically acceptable medium, that is to say a medium compatible with the skin, mucous membranes, hair and scalp.

The aqueous composition can also comprise fatty substances. The term “fatty substances” is understood to mean any water-insoluble compound, it being possible for this compound to be an oil (liquid fatty substance) or a solid fatty substance. The composition is then provided in the form of a dispersion or emulsion.

The aqueous composition can thus be provided in the form of solutions, of more or less fluid emulsions (lotions or creams, depending on the viscosity), for example of water-in-oil (W/O) or oil-in-water (O/W) or multiple (W/O/W or O/W/O) emulsions chosen in particular from conventional emulsions or specific emulsions, such as, for example, from:
O/W emulsions based on oily globules provided with a lamellar liquid crystal coating, such as described in the documents EP-A-641 557 and EP-A-705 593;
surfactant-free O/W emulsions stabilized by water-dispersible anionic polymers, such as those described in the document EP-A-864 320;
O/W emulsions based on polymers derived from 2-acrylamido-2-methylpropanesulphonic acid (AMPS polymer), such as described in the document EP-A-815 844;
O/W emulsions stabilized by hydrophobic AMPS polymers, as described in the documents EP-A-1 069 142, WO-A-2002/43689, WO-A-2002/44231, WO-A-2002/44271, WO-A-2002/44270, WO-A-2002/43686, WO-A-2002/44267, WO-A-2002/43688, WO-A-2002/43677, WO-A-2002/43687 and WO-A-2002/44230;
fluid emulsions based on thermoassociative polymers, such as described in the documents EP-A-1 355 990, EP-A-1 355 625, EP-A-1 307 501 and EP-A-1 363 964;
O/W emulsions obtained by the PIT method (emulsion obtained by phase inversion. PIT=Phase Inversion Temperature), such as described in documents WO-A-89/11907, DE-A-4318171 and EP-A-815 846;
nanoemulsions, such as those described in patent applications EP-A-728 460, EP-A-780 114, EP-A-780 115, EP-A-879 589, EP-A-1 010 413, EP-A-1 010 414, EP-A-1 010 415, EP-A-1 010 416, EP-A-1 013 338, EP-A-1 016 453, EP-A-1 018 363, EP-A-1 020 219, EP-A-1 025 898, EP-A-1 120 102, EP-A-1 120 101, EP-A-1 160 005, EP-A-1 172 077 and EP-A-1 353 629.

The aqueous composition can also be in the form of foaming aqueous products, such as described, for example, in the documents EP-A-1 166 747, EP-A-1 172 096, EP-A-1 172 095, EP-A-1 174 122, EP-A-1 277 463, EP-A-1 295 594 and FR-A-2 824 262.

According to a specific embodiment of the invention, the aqueous composition used according to the invention is an emulsion. The proportion of the oily phase of the emulsion can range from 1 to 80% by weight and preferably from 1 to 50% by weight, with respect to the total weight of the composition. The oils, and the emulsifiers and coemulsifiers possibly present, used in the composition in the emulsion form are chosen from those conventionally used in the cosmetic or dermatological field. The emulsifier and the coemulsifier, when they are present, are generally present in a proportion ranging from 0.2 to 30% by weight, preferably from 0.3 to 20% by weight and better still from 0.5 to 15% by weight, with respect to the total weight of the composition. The emulsion can additionally comprise lipid vesicles.

The emulsions generally comprise at least one emulsifier chosen from amphoteric, anionic, cationic or nonionic emulsifiers, used alone or as a mixture. The emulsifiers are appropriately chosen according to the continuous phase of the emulsion to be obtained (W/O or O/W). When the emulsion is a multiple emulsion, it generally comprises an emulsifier in the primary emulsion and an emulsifier in the external phase into which the primary emulsion is introduced.

Mention may be made, as emulsifiers which can be used in the preparation of the W/O emulsions, for example, of sorbitan, glycerol or sugar alkyl esters or ethers; silicone surfactants, such as dimethicone copolyols, for example the mixture of cyclomethicone and of dimethicone copolyol sold under the names DC 5225 C and DC 3225 C by Dow Corning, and such as alkyl dimethicone copolyols, for example the lauryl dimethicone copolyol sold under the name “Dow Corning 5200 Formulation Aid” by Dow Corning, the cetyl dimethicone copolyol sold under the name Abil EM 90® by Goldschmidt and the polyglyceryl-4 isostearate/cetyl dimethicone copolyol/hexyl laurate mixture sold under the name Abil WE 09® by Goldschmidt. It is also possible to add thereto one or more coemulsifers which can advantageously be chosen from the group consisting of branched-chain fatty acid and polyol esters and in particular branched-chain fatty acid and glycerol and/or sorbitan esters, for example polyglyceryl isostearate, such as the product sold under the name Isolan GI 34 by Goldschmidt, sorbitan isostearate, such as the product sold under the name Arlacel 987 by ICI, and sorbitan glyceryl isostearate, such as the product sold under the name Arlacel 986 by ICI, and their mixtures.

Mention may be made, as emulsifiers which can be used in the preparation of the O/W emulsions, for example, of nonionic emulsifiers, such as esters of fatty acids and of oxyalkylene (more particularly polyoxyethylene) polyols, for example polyethylene glycol stearates, such as PEG-100 stearate, PEG-50 stearate and PEG-40 stearate; and their mixtures, such as the mixture of glyceryl monostearate and of polyethylene glycol (100 EO) stearate sold under the name Simulsol 165 by Seppic; oxyalkylenated esters of fatty acids and of sorbitan comprising, for example, from 20 to 100 EO units, for example those sold under the trade names Tween 20 or Tween 60 by Uniqema; oxyalkylenated (oxyethylenated and/or oxypropylenated) ethers of fatty alcohols; alkoxylated or nonalkoxylated esters of sugars, such as sucrose stearate and such as PEG-20 methyl glucose sesquistearate; sorbitan esters, such as the sorbitan palmitate sold under the name Span 40 by Uniqema; esters of diacid and of fatty alcohol, such as dimyristyl tartrate; mixtures of these emulsifiers, such as, for example, the mixture of glyceryl stearate and of PEG-100 stearate sold under the name Arlacel 165 by Uniqema; and mixtures comprising these emulsifiers, such as the mixture of dimyristyl tartrate, of cetearyl alcohol, of pareth-7 and of PEG-25 laureth-25 sold under the name Cosmacol PSE by Sasol (INCI name: dimyristyl tartrate/cetearyl alcohol/12-15 pareth 7/PPG 25 laureth 25).

Coemulsifiers can be added to these emulsifiers, such as, for example, fatty alcohols having from 8 to 26 carbon atoms, such as cetyl alcohol, stearyl alcohol and their mixture (cetearyl alcohol), octyldodecanol, 2-butyloctanol, 2-hexyldecanol, 2-undecylpentadecanol or oleyl alcohol, or fatty acids.

It is also possible to prepare emulsions devoid of emulsifying surfactants or comprising less than 0.5% of the total weight of the composition thereof by using appropriate compounds, for example polymers having emulsifying properties, such as the polymers sold under the names Carbopol 1342 and Pemulen by Noveon; or polymers in emulsion, such as that sold under the name Sepigel 305 by Seppic (INCI name: polyacrylamide/C13-C14 isoparaffin/laureth-7); particles formed of ionic or nonionic polymers, more particularly particles formed of anionic polymer, such as in particular polymers of isophthalic acid or of sulphoisophthalic acid, in particular the phthalate/sulphoisophthalate/glycol copolymers (for example diethylene glycol/phthalate/isophthalate/1,4-cyclohexanedimethanol (INCI name: diglycol/CHDM/isophthalates/SIP copolymer)) sold under the names Eastman AQ polymer (AQ35S, AQ38S, AQ55S, AQ48 Ultra) by Eastman Chemical. It is also possible to prepare emulsions devoid of emulsifiers which are stabilized by silicone particles or particles of metal oxide, such as TiO₂, or others.

When the aqueous composition is in the form of an emulsion, it comprises at least one oily phase which comprises at least one oil, in particular a cosmetic oil. The term “oil” is understood to mean a fatty substance which is liquid at ambient temperature (25° C.).

Mention may be made, as oils which can be used in the composition of the invention, for example, of:
hydrocarbon oils of animal origin, such as perhydrosqualene (or squalane);
hydrocarbon oils of vegetable origin, such as liquid triglycerides of fatty acids comprising from 4 to 10 carbon atoms, such as triglycerides of heptanoic acid or octanoic acid, or also oils of vegetable origin, for example sunflower, maize, soybean, cucumber, grape seed, sesame, hazelnut, apricot, macadamia, arara, coriander, castor or avocado oils, jojoba oil, or shea butter oil, or also triglycerides of caprylic/capric acids, such as those sold by Stearineries Dubois or those sold under the trade names Miglyol 810, 812 and 818 by Dynamit Nobel;
synthetic esters and ethers, in particular of fatty acids, such as oils of formulae R¹COOR² and R¹OR² in which R¹ represents the residue of a fatty acid comprising from 8 to 29 carbon atoms and R² represents a linear or branched hydrocarbon chain comprising from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate, heptanoates, octanoates or decanoates of fatty alcohols; polyol esters, such as propylene glycol dioctanoate, neopentyl glycol diheptanoate and diethylene glycol diisononanoate; pentaerythritol esters, such as pentaerythrityl tetraisostearate; lipophilic derivatives of amino acids, such as isopropyl lauroyl sarcosinate (INCI name), sold under the name Eldew SL 205 by Ajinomoto;
linear or branched hydrocarbons of mineral or synthetic origin, such as mineral oils (mixture of hydrocarbon oils derived from oil; INCI name: mineral oil), volatile or nonvolatile liquid paraffins and their derivatives, liquid petrolatum, polydecenes, isohexadecane, isododecane, hydrogenated isoparaffin, such as Parleam® oil, sold by NOF Corporation (INCI name; hydrogenated polyisobutene);
silicone oils, such as volatile or nonvolatile polymethylsiloxanes (PDMSs) comprising a linear or cyclic silicone chain which are liquid or pasty at ambient temperature, in particular cyclopolydimethylsiloxanes (cyclomethicones), such as cyclopentasiloxane and cyclohexadimethylsiloxane; polydimethylsiloxanes comprising pendent alkyl, alkoxy or phenyl groups or alkyl, alkoxy or phenyl groups at the end of the silicone chain, which groups have from 2 to 24 carbon atoms; phenylated silicones, such as phenyl trimethicones, phenyl dimethicones, phenyl(trimethylsiloxy)diphenylsiloxanes, diphenyl dimethicones, diphenyl(methyldiphenyl)trisiloxanes, (2-phenylethyl)trimethylsiloxysilicates and polymethylphenylsiloxanes;
fluorinated oils, such as those which partially comprise hydrocarbon and/or silicone, such as those disclosed in document JP-A-2-295912;
ethers, such as dicaprylyl ether (INCI name); and benzoates of C₁₂-C₁₅ fatty alcohols (Finsolv TN from FINETEX);
their mixtures.

The aqueous composition and/or the film or films can additionally comprise any appropriate additive compound, such as, for example, formulation adjuvants and/or active principles.

Active Principles

The water-soluble anhydrous film or the aqueous composition or both can comprise at least one active principle. One of the advantages of the combination according to the invention comprising the film or films and the aqueous composition is, for example, to be able to obtain a cosmetic product comprising incompatible active principles, one or more compatible active principles being present in the aqueous composition and one or more different active principles incompatible with those of the aqueous composition being present in the anhydrous film or films. The mixing at the time of use of the aqueous composition and of the film or films then results in a product comprising incompatible active principles, their incompatibility not presenting a disadvantage since the product is used immediately. This can also make it possible to be able to obtain products having higher concentrations of active principles than the concentrations which might be obtained using just one composition.

The term “active principle” is understood to mean any compound having a beneficial effect on the keratinous substance on which the final product is applied, in particular on the skin.

Mention may be made, as active principles having a cosmetic or dermatological application, as examples and without this list being limiting, of:

I) moisturizing active principles, such as, for example, sodium lactate; polyols, in particular glycerol, sorbitol or polyethylene glycols; mannitol; amino acids; hyaluronic acid; lanolin; urea and mixtures comprising urea, such as NMF (natural moisturizing factor); urea derivatives, in particular (hydroxyalkyl)ureas, such as the (hydroxyethyl)urea available commercially in the form of a 50′ by weight mixture in water from National Starch under the trade name Hydrovance®; petrolatum; salts and derivatives of pyrrolidonecarboxylic acid, such as N-lauroyl-pyrrolidonecarboxylic acid; essential fatty acids; essential oils; and their mixtures.

II) Antiageing active principles which can be chosen from any active principle capable of treating or preventing any sign of ageing of the skin. They can be chosen, for example, from agents for combating free radicals, keratolytic agents, vitamins, anti-elastase and anti-collagenase agents, proteins, fatty acid derivatives, steroids, trace elements, algal and plankton extracts, enzymes and coenzymes, flavonoids, ceramides, muscle relaxants, glycosides capable of stimulating the synthesis of glycosaminoglycans, and their mixtures.

1) Mention may in particular be made, as agents for combating free radicals and antioxidants, of phosphonic acid derivatives, such as ethylenediamine-tetra(methylenephosphonic acid), hexamethylenediamine-tetra(methylenephosphonic acid), diethylenetriamine-penta(methylenephosphonic acid) and their salts, in particular their sodium salts; ethylenediaminetetraacetic acid and its salts, such as the sodium salt; guanosine; superoxide dismutase; tocopherol (vitamin E) and its derivatives (acetate); ethoxyquin; lactoferrin; lactoperoxidase, and nitroxide derivatives; glutathione peroxidase; plant extracts which are active in combating free radicals, such as the aqueous extract of wheatgerm sold by Silab under the reference Detoxiline; green tea; and their mixtures.

2) Mention may be made, as keratolytic agents, for example, of α-hydroxy acids, in particular acids derived from fruits, such as glycolic acid, lactic acid, malic acid, citric acid, tartaric acid or mandelic acid, their derivatives and their mixtures; β-hydroxy acids, such as salicylic acid and its derivatives, for example 5-(n-octanoyl)salicylic acid or 5-(n-dodecanoyl)salicylic acid; α-keto acids, such as ascorbic acid or vitamin C and its derivatives, for example its salts, such as sodium ascorbate, magnesium ascorbyl phosphate or sodium ascorbyl phosphate; its esters, such as ascorbyl acetate, ascorbyl palmitate and ascorbyl propionate, or its sugars, such as glycosylated ascorbic acid, and their mixtures; β-keto acids; retinoids, such as retinol (vitamin A) and its esters, retinal, retinoic acid and its derivatives, and also the retinoids described in the documents FR-A-2 570 377, EP-A-199 636, EP-A-325 540 and EP-A-402 072; adapalene; carotenoids; and their mixtures.

3) Mention may in particular be made, as vitamins, in addition to vitamins A, E and C indicated above, of vitamin B3 (or vitamin PP or niacinamide) and its derivatives (tocopherol nicotinate, esters of nicotinyl alcohol and of carboxylic acids, 2-chloronicotinamide, 6-methylnicotinamide, 6-aminonicotinamide, N-methylnicotinamide, N,N-dimethylnicotinamide, N-(hydroxymethyl)nicotinamide, quinolinic acid imide, nicotinanilide, N-benzylnicotinamide, N-ethylnicotinamide, nifenazone, nicotinaldehyde, isonicotinic acid, methylisonicotinic acid, thionicotinamide, nialamide, 2-mercaptonicotinic acid, nicomol and niaprazine); vitamin B5 (or panthenol or panthenyl alcohol or 2,4-dihydroxy-N-(3-hydroxypropyl)-3,3-dimethylbutanamide), in its various forms: D-panthenol, DL-panthenol) and its derivatives and analogues, such as calcium pantothenate, pantethine, pantetheine, panthenyl ethyl ether, pangamic acid, pyridoxine, pantoyl lactone, and natural compounds comprising them, such as royal jelly; vitamin D and its analogues, such as those described in the document WO-A-00/26167; vitamin F or its analogues, such as mixtures of unsaturated acids having at least one double bond and in particular mixtures of linoleic acid, of linolenic acid and of arachidonic acid, or compounds comprising them and in particular the oils of vegetable origin comprising them, such as, for example, jojoba oil, and their mixtures.

4) Mention may in particular be made, as anti-elastase agents, of peptide derivatives and in particular peptides from seeds of leguminous plants, such as those sold by Laboratoires Seriobiologiques de Nancy under the reference Parelastyl; the N-acylamino amide derivatives described in Patent Application FR-A-2 180 033, such as, for example, ethyl {2-[acetyl(3-(trifluoromethyl)phenyl)amino]-3-methylbutyrylamino}acetate and {2-[acetyl(3-(trifluoromethyl)phenyl)amino]-3-methylbutyrylamino}acetic acid, and their mixtures.

5) Mention may be made, as anti-collagenase agents, of metalloprotease inhibitors, such as ethylenediamine-tetraacetic acid (EDTA), cysteine, and their mixtures.

6) Mention may be made, as proteins, for example, of wheat or soybean proteins, their hydrolysates, such as those sold by Silab under the reference Tensine, and their mixtures.

7) Mention may in particular be made, as fatty acid derivatives, of polyunsaturated phospholipids, including the phospholipids of octopus essential fatty acids, and their mixtures.

8) Mention may be made, as steroids, for example, of DHEA or dehydroepiandrosterone, its biological precursors, its metabolites and their mixtures. The term “biological precursors” of DHEA is understood to mean in particular Δ5-pregnenolone, 17α-hydroxypregnenolone and 17α-hydroxypregnenolone sulphate. The term “DHEA derivatives” is understood to mean both its metabolic derivatives and its chemical derivatives. Mention may in particular be made, as metabolic derivatives, of Δ5-androstene-3,17-diol and in particular 5-androstene-3β,17β-diol, Δ4-androstene-3,17-dione, 7-hydroxy-DHEA (7α-hydroxy-DHEA or 7β-hydroxy-DHEA), 7-keto-DHEA, which is itself a metabolite of 7β-hydroxy-DHEA, and benzoyl DHEA.

9) Mention may be made, as trace elements, for example, of copper, zinc, selenium, iron, magnesium, manganese and their mixtures.

10) Mention may be made, as algal extracts, of extracts of red or brown algae, for example the extract of brown algae of the Laminaria genus, such as extracts of the species Laminaria digitata, and more particularly that sold by Codif under the name Phycosaccharides, which is a concentrated solution of an oligosaccharide comprising a sequence of two uric acids: mannuronic acid and guluronic acid.

11) Mention may be made, as extracts of planktons, of the plankton in aqueous dispersion (INCI name: Vitreoscilla Ferment) sold under the name Mexoryl SAH by Chimex.

12) Use may be made, as enzymes, of any enzyme of animal origin, microbiological origin (bacterial, fungal or viral) or synthetic origin (obtained via chemical or biotechnological synthesis), in the pure crystalline form or in a form diluted in an inert diluent. Mention may be made, for example, of lipases, proteases, phospholipases, laccases, cellulases, peroxidases, in particular lactoperoxidases, catalases, superoxide dismutases, or plant extracts comprising the abovementioned enzymes, and their mixtures. They can be chosen, for example, from that sold under the trade name “Subtilisine SP 554” by Novo Nordisk and that sold under the trade name “Lysoveg LS” by Laboratoires Sérobiologiques de Nancy.

13) Use may in particular be made, as coenzymes, of ubiquinone or coenzyme Q10, which belongs to the family of benzoquinones comprising an alkylene chain, or coenzyme R, which is biotin (or vitamin H), and their mixtures.

14) Mention may be made, as flavonoids, for example, of isoflavonoids, which constitute a subclass of the flavonoids, formed of a 3-phenylchroman backbone, which can comprise various substituents and different levels of oxidation. The term “isoflavonoid” combines several classes of compounds, among which may be mentioned isoflavones, isoflavanones, rotenoids, pterocarpans, isoflavans, isoflavan-3-enes, 3-arylcoumarins, 3-aryl-4-hydroxycoumarins, coumestans, coumaronochromones, methyldeoxybenzoins, 2-arylbenzofurans, and their mixtures. The isoflavonoids can be of natural or synthetic origin. The term “natural origin” is understood to mean the isoflavonoid, in the pure state or in solution at different concentrations, obtained by various extraction processes from a component, generally a plant, of natural origin. The term “synthetic origin” is understood to mean the isoflavonoid, in the pure state or in solution at different concentrations, obtained by chemical synthesis. Mention may be made, as isoflavonoids of natural origin, of daidzin, genistin, daidzein, formononetin, cuneatin, genistein, isoprunetin and prunetin, cajanin, orobol, pratensein, santal, junipegenin A, glycitein, afrormosin, retusin, tectorigenin, irisolidone, jamaicin, and their analogues and metabolites.

15) Use may be made, as ceramides, of any type of ceramide of natural or synthetic origin, for example of type II, of type III, of type IV, of type V or of type VI, and their mixtures. Mention may be made, for example, as ceramides, of N-oleoyldihydrosphingosine, N-stearoylphytosphingosine, N-hydroxybehenoyldihydrosphingosine, N-hydroxypalmitoyldihydrosphingosine, N-linoleoyldihydrosphingosine, N-palmitoyldihydrosphingosine, N-stearoyldihydrosphingosine, N-behenoyldihydrosphingosine, and their mixtures.

16) Mention may be made, as muscle relaxants, which are agents for smoothing out expression wrinkles, for example, of sapogenins (see document EP-A-1 352 643); adenosine and its derivatives (see document FR-0 214 828); antagonists of receptors associated with calcium channels (see document FR-A-2 793 681), in particular manganese and its salts (see document FR-A-2 809 005) and alverine (see document FR-A-2 798 590); antagonists of receptors associated with chlorine channels, including glycine (see document EP-A-0 704 210) and some Iris pallida extracts (see document FR-A-2 746 641).

17) Mention may in particular be made, as C-glycoside derivatives capable of stimulating the synthesis of glycosoaminoglycans, of the C-glycoside derivatives of the following formula: 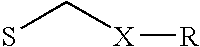
in which:
S represents a monosaccharide or polysaccharide of up to 20 sugar units, in the pyranose and/or furanose form and of the L and/or D series, the mono- or polysaccharide exhibiting at least one hydroxyl functional group which has to be free and/or optionally one or more amine functional groups which may be protected,
the S—CH₂X bond represents a bond of C-anomeric nature,
X represents a group chosen from: —CO—, —CH(OH)—, —CH(NR₁R₂)—, —CHR′— or —C(═CHR′)—,
R represents a saturated or unsaturated, linear or branched, alkyl, perfluoroalkyl or hydrofluoroalkyl chain, a cycloalkyl, cycloperfluoroalkyl or cyclohydrofluoroalkyl ring, comprising from 1 to 18 carbon atoms, a phenyl or benzyl radical, it being possible for the chain, the ring or the radical optionally to be interrupted by one or more heteroatoms chosen from oxygen, sulphur, nitrogen or silicon and optionally to be substituted by at least one radical chosen from —OR′₁, —SR″₁, —NR′″₁—R′₂, —COOR″₂, —CONHR′″₂, —CN, halogen, perfluoroalkyl or hydrofluoroalkyl and/or at least one cycloalkyl, aryl or heterocyclic radical, which are optionally substituted, where
R′, R₁ and R₂, which are identical or different, have the same definition as that given for R and can also represent a hydrogen or a hydroxyl radical,
R′₁, R′₂, R″₁, R″₂, R′″₁ and R′″₂, which are identical or different, represent a hydrogen atom or a radical chosen from a saturated or unsaturated, linear or branched, alkyl, hydroxyl, perfluoroalkyl and/or hydrofluoroalkyl radical comprising from 1 to 30 carbon atoms,
   and in particular the compounds described in the document EP-A-1 345 919.

III) Whitening or depigmenting agents, such as, for example, kojic acid and its derivatives; hydroquinone and its derivatives, such as arbutin and its esters; vitamin C and its derivatives, such as magnesium ascorbyl phosphate; salts, such as calcium D-pantetheinesulphonate; ellagic acid and its derivatives; rucinol; linoleic acid and its derivatives; plant extracts, in particular liquorice, mulberry or skullcap extracts; glutathione and its precursors; cysteine and its precursors; the compounds derived from aminophenol described in the document WO-A-99/10318, such as, in particular, N-ethyloxycarbonyl-4-aminophenol, N-ethyloxycarbonyl-O-ethyloxycarbonyl-4-aminophenol, N-cholesteryloxycarbonyl-4-aminophenol or N-ethylaminocarbonyl-4-aminophenol; and mixtures of these compounds.

(IV) Skin-colouring agents, such as dihydroxyacetone (DHA), natural dyes, such as plant extracts, such as, for example, sorghum extracts, and also optical brighteners, such as the distyryl-4,4′-biphenyldisulphonate sold by Ciba-Geigy under the name of Tinopal CBS-X®. Mention may also be made, for example, of the sodium 4,4′-bis[(4,6-dianilino-1,3,5-triazin-2-yl)amino]stilbene-2,2′-disulphonate and the 2,5-thiophenediylbis(5-(tert-butyl)-1,3-benzoxazole) sold by Ciba-Geigy under the names of Tinopal SOP® and Uvitex OB®.

V) Sunscreens, which can be chosen from the UV-A and UV-B chemical screening agents or the physical screening agents commonly used in the cosmetics field.

Mention may be made, as UV-B screening agents, for example, of:

(1) salicylic acid derivatives, in particular homomethyl salicylate and octyl salicylate;

(2) cinnamic acid derivatives, in particular 2-ethylhexyl p-methoxycinnamate, sold by Givaudan under the name Parsol MCX;

(3) the liquid β,β′-diphenylacrylate derivatives, in particular 2-ethylhexyl α-cyano-α,β′-diphenylacrylate or octocrylene, sold by BASF under the name Uvinul N539;

(4) p-aminobenzoic acid derivatives;

(5) 4-methylbenzylidenecamphor, sold by Merck under the name Eusolex 6300;

(6) 2-phenylbenzimidazole-5-sulphonic acid, sold under the name Eusolex 232 by Merck;

(7) 1,3,5-triazine derivatives, in particular:
2,4,6-tris[p-(2′-ethylhexyl-1′-oxycarbonyl)anilino]-1,3,5-triazine, sold by BASF under the name Uvinul T150, and
dioctylbutamidotriazone, sold by the company Sigma 3V under the name Uvasorb HEB;.

(8) the mixtures of these screening agents.

Mention may be made, as UV-A screening agents, for example, of:

(1) dibenzoylmethane derivatives, in particular 4-(tert-butyl)-4′-methoxydibenzoylmethane, sold by Givaudan under the name Parsol 1789;

(2) benzene-1,4-di(3-methylidenecamphor-10-sulphonic acid), optionally in the partially or completely neutralized form, sold under the name Mexoryl SX by Chimex;

(3) benzophenone derivatives, for example:
2,4-dihydroxybenzophenone (benzophenone-1);
2,2′,4,4′-tetrahydroxybenzophenone (benzophenone-2);
2-hydroxy-4-methoxybenzophenone (benzophenone-3), sold under the name Uvinul M40 by BASF;
2-hydroxy-4-methoxybenzophenone-5-sulphonic acid (benzophenone-4) and also its sulphonate form (benzophenone-5), sold by BASF under the name Uvinul MS40;
2,2′-dihydroxy-4,4′-dimethoxybenzophenone (benzophenone-6);
5-chloro-2-hydroxybenzophenone (benzophenone-7);
2,2′-dihydroxy-4-methoxybenzophenone (benzophenone-8);
the disodium salt of 2,2′-dihydroxy-4,4′-dimethoxybenzophenone-5,5′-disulphonic diacid (benzophenone-9);
2-hydroxy-4-methoxy-4′-methylbenzophenone (benzophenone-10);
benzophenone-11;
2-hydroxy-4-(octyloxy)benzophenone (benzophenone-12).

(4) silane derivatives or polyorganosiloxanes comprising a benzophenone group;

(5) anthranilates, in particular menthyl anthranilate, sold by Haarman & Reimer under the name Neo Heliopan MA;

(6) compounds comprising, per molecule, at least two benzazolyl groups or at least one benzodiazolyl group, in particular 1,4-bis(benzimidazolyl)phenylene-3,3′,5,5′-tetrasulphonic acid and also its salts, sold by Haarman & Reimer;

(7) silicon derivatives of N-substituted benzimidazolylbenzazoles or of benzofuranylbenzazoles, in particular:
2-[1-[3-[1,3,3,3-tetramethyl-1-[(trimethylsilyl)oxy]-disiloxanyl]propyl]-1H-benzimidazol-2-yl]benzoxazole;
2-[1-[3-[1,3,3,3-tetramethyl-1-[(trimethylsilyl)oxy]-disiloxanyl]propyl]-1H-benzimidazol-2-yl]benzothiazole;
2-[1-(3-(trimethylsilanyl)propyl)-1H-benzimidazol-2-yl]benzoxazole;
6-methoxy-1,1′-bis(3-(trimethylsilanyl)propyl) -1H,1′H-[2,2′]bibenzimidazolylbenzoxazole;
2-[1-(3-(trimethylsilanyl)propyl)-1H-benzimidazol-2-yl]benzothiazole,
   which are described in the document EP-A-1 028 120;

(8) triazine derivatives, in particular 2,4-bis[4-(2-ethylhexyloxy)-2-hydroxyphenyl]-6-(4-methoxyphenyl)-1,3,5-triazine, sold by Ciba-Geigy under the name Tinosorb S, and 2,2′-methylenebis[6-(2H-benzotriazol-2-yl)-4-(1,1,3,3-tetramethylbutyl)phenol], sold by Ciba-Geigy under the name Tinosorb M;

(9) benzotriazole silicones, which are described in particular in the document EP-A-0 392 883, especially the benzotriazole silicone of formula: 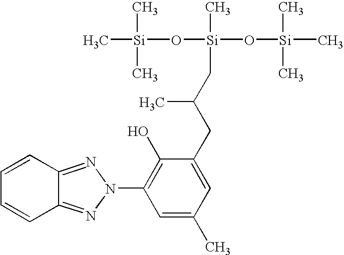
(10) their mixtures.

Use may also be made of a mixture of several of these screening agents.

Mention may be made, as physical screening agents, of oxides of titanium (amorphous titanium dioxide or titanium dioxide crystallized in the rutile and/or anatase form), of zinc, of iron, of zirconium or of cerium or their mixtures. These metal oxides can be in the form of particles having a micrometric or nanometric size (nanopigments). In the form of nanopigments, the mean sizes of the particles range, for example, from 5 to 100 nm. Use is preferably made of nanopigments.

VI) Concealing agents, such as vitamin K1 and its derivatives.

VII) Antimicrobial, antifungal or antiacne active principles, in particular hydroxy acids, such as salicylic acid and its derivatives, glycolic acid, lactic acid, ellagic acid, 2,4,4′-trichloro-2′-hydroxydiphenyl ether (or triclosan), 3,4,4′-trichlorocarbanilide (or triclocarban), phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulphaconazole, sulconazole, terbinafine, ciclopirox, ciclopirox olamine, undecylenic acid and its salts, benzoyl peroxide, 3-hydroxybenzoic acid, 4-hydroxybenzoic acid, phytic acid, N-acetyl-L-cysteine, lipoic acid, azelaic acid and its salts, arachidonic acid, resorcinol, octopirox, octoxyglycerol, octanoylglycine, caprylyl glycol, 10-hydroxy-2-decanoic acid, dichlorophenyl imidazole dioxolane and its derivatives described in the document WO-A-93/18743, farnesol, phytosphingosines, selenium derivatives, zinc pyrithione, tetracyclins, such as erythromycin, and the mixtures of these compounds.

VIII) Antiseborrhoeic agents, for example chosen from:
sulphur and sulphur derivatives;
zinc salts, such as zinc lactate, gluconate, pidolate, carboxylate, salicylate and/or cysteate;
selenium chloride;
vitamin B6 or pyridoxine;
the mixture of capryloylglycine, of sarcosine and of Cinnamomum zeylanicum extract sold in particular by Seppic under the trade name Sepicontrol A5®;
a Laminaria saccharina extract sold in particular by Secma under the trade name Phlorogine®;
a Spiraea ulmaria extract sold in particular by Silab under the trade name Sebonormine®;
plant extracts of the species Arnica montana, Cinchona succirubra, Eugenia caryophyllata, Humulus lupulus, Hypericum perforatum, Mentha piperita, Rosmarinus officinalis, Salvia oficinalis and Thymus vulgaris, all sold, for example, by Maruzen;
a Serenoa repens extract sold in particular by Euromed;
extracts of plants of the genus Silybum;
plant extracts comprising sapogenins and in particular extracts of Dioscoreae plants rich in diosgenin or hecogenin;
Eugenia caryophyllata extracts comprising eugenol and eugenyl glucoside;
   and their mixtures.
   IX) Antiperspirant and deodorant agents:

Mention may be made, as antiperspirant agents, for example, of aluminium and/or zirconium salts, such as, for example, aluminium chlorohydrate, aluminium chlorohydrex, aluminium chlorohydrex PEG, aluminium chlorohydrex PG, aluminium dichlorohydrate, aluminium dichlorohydrex PEG, aluminium dichlorohydrex PG, aluminium sesquichlorohydrate, aluminium sesquichlorohydrex PEG, aluminium sesquichlorohydrex PG, alum salts, aluminium sulphate, aluminium zirconium octachlorohydrate, aluminium zirconium pentachlorohydrate, aluminium zirconium tetrachlorohydrate, aluminium zirconium trichlorohydrate and more particularly the aluminium chlorohydrate sold by Reheis under the name Reach 301 or 303 or by Guilini Chemie under the name Aloxicoll PF 40 and the aluminium and zirconium salt sold by Reheis under the name Reach AZP-908-SUF; complexes of zirconium hydroxychloride and of aluminium hydroxychloride with an amino acid, such as those described in the document U.S. Pat. No. 3,792,068, commonly known under the name “ZAG complexes”, such as, for example, aluminium zirconium octachlorohydrex GLY, aluminium zirconium pentachlorohydrex GLY, aluminium zirconium tetrachlorohydrate GLY and aluminium zirconium trichlorohydrate GLY.

Mention may be made, as deodorant agents, for example, of zinc pyrrolidonecarboxylate (more commonly known as zinc pidolate), zinc sulphate, zinc chloride, zinc lactate, zinc gluconate and zinc phenolsulphonate, 2,4,4′-trichloro-2′-hydroxydiphenyl ether (triclosan), 2,4-dichloro-2′-hydroxydiphenyl ether, 3′,4′,5′-trichlorosalicylanilide, 1-(3′,4′-dichlorophenyl)-3-(4′-chlorophenyl)urea (triclocarban) or 3,7,11-trimethyldodeca-2,5,10-trienol (farnesol); quaternary ammonium salts, such as cetyltrimethylammonium salts or cetylpyridinium salts; chlorhexidine and its salts; diglycerol monocaprate, diglycerol monolaurate, glycerol monolaurate; polyhexamethylene biguanide salts.

X) Soothing active principles, such as pentacyclic triterpenes and plant extracts (e.g.: Glycyrrhiza glabra) comprising them, such as β-glycyrrhetinic acid and its salts and/or its derivatives (glycyrrhetinic acid monoglucuronide, stearyl glycyrrhetinate, 3-stearoyloxyglycyrrhetic acid); ursolic acid and its salts; oleanolic acid and its salts; betulinic acid and its salts; extracts of Paeonia suffruticosa or lactiflora, of Laminaria saccharina, of chamomile, of Pygeum, of Boswellia serrata, of Centipeda cunninghami, of Helianthus annuus, of Linum usitatissimum, of Cola nitida, of clove, of Epilobium angustifolium or Bacopa monnieri; salicylic acid salts and in particular zinc salicylate; phycosaccharides from Codif; canola oil; bisabolol; allantoin; Sepivital EPC (phosphoric diester of vitamin E and C) from Seppic; omega-3 unsaturated oils, such as musk rose oil, blackcurrant oil, Echium oil or fish oil; capryloylglycine; Seppicalm VG (sodium palmitoylproline and Nymphaea alba) from Seppic; tocotrienols; piperonal; aloe vera; phytosterols.

XI) Lipolytic or slimming active principles, that is to say having a favourable activity, direct or indirect, on reducing adipose tissue, such as:
xanthine derivatives, such as caffeine and its derivatives, in particular the 1-hydroxyalkylxanthines described in the document FR-A-2 617 401, caffeine citrate, theophylline and its derivatives, theobromine, acefylline, aminophylline, chloroethyltheophylline, diprophylline, diniprophylline, etamiphylline and its derivatives, etophylline or proxyphylline; or the combinations comprising xanthine derivatives, such as the combination of caffeine and of silanol (methylsilanetriol derivative of caffeine), for example the product sold by Exsymol under the name cafeisilane C; or else compounds of natural origin comprising xanthine bases, in particular caffeine, such as extracts of tea, of coffee, of guarana, of mate, of cola (Cola nitida) and in particular the dry extract of guarana (Paullinia sorbillis) fruit comprising 8 to 10% of caffeine; ephedrine and its derivatives, which can in particular be found in the natural state in plants such as ma huang (ephedra plant);
plant extracts and extracts of marine origin which are either active with regard to the receptors to be inhibited, such as β-2 blockers or NPY blockers (described in the document EP-A-838 217), or inhibit the synthesis of LDL or VLDL receptors, or are active in stimulating β receptors and G proteins, resulting in the activation of adenylyl cyclase. Mention may be made, as plant extracts of this type, for example, of:
extracts of Garcinia cambogia,
extracts of Bupleurum chinensis,
extracts of English ivory (Hedera helix), of arnica (Arnica montana L.), of rosemary (Rosmarinus officinalis N.), of marigold (Calendula officinalis), of sage (Salvia officinalis L.), of ginseng (Panax ginseng), of St. John's wort (Hypericum perforatum), of butcher's broom (Ruscus aculeatus L.), of meadowsweet (Filipendula ulmaria L.), of Java tea (Orthosiphon stamineus Benth.), of birch (Betula alba), of cecropia and of argan,
extracts of ginkgo biloba,
extracts of field horsetail,
extracts of horse chestnut,
extracts of cang zhu,
   extracts of Chrysanthellum indicum,
extracts of Dioscoreae plants rich in diosgenin or pure diosgenin or hecogenin and their derivatives,
extracts of plants of the genus Armeniacea, Atractylodis, Platycodon, Sinom-menum, Pharbitidis or Flemingia,
extracts of Coleus, such as C. forskohlii, C. blumei, C. esquirolii, C. scutellarioides, C. xanthantus and C. barbatus, such as the extract of Coleus barbatus root comprising 60% of forskolin,
extracts of Ballota,
extracts of Guioa, of Davallia, of Terminalia, of Barringtonia, of Trema or of Antirobia.
   Mention may be made, as extracts of marine origin, of:
extracts of algae or of phytoplankton, such as rhodysterol or the extract of Laminaria digitata sold under the name Phycox 75 by Secma, the skeletonema alga described in the document FR-A-2 782 921 or the diatoms described in the document FR-A-2 774 292.

XII) Tightening agents

Mention may be made, as tightening agents, for example, of:
synthetic polymers;
polymers of natural origin;
mixed silicates;
colloidal particles formed of inorganic fillers.

The synthetic polymers which can be used as tightening agent can be chosen from:
polyurethane polymers and copolymers;
acrylic polymers and copolymers;
grafted silicone polymers;
water-soluble or water-dispersible polymers comprising water-soluble or water-dispersible units and units comprising an LCST (lower critical solution temperature), such as the polymers described in the document FR-A-2 819 429.

The polymers of natural origin which can be used as tightening agent can be chosen from:
plant proteins and plant protein hydrolysates;
polysaccharides of vegetable origin, optionally in the form of microgels, such as starch;
latexes of vegetable origin.

The mixed silicates can be of natural or synthetic origin and they can include several types of cations chosen from alkali metals (for example, Na, Li or K) or alkaline earth metals (for example, Be, Mg or Ca) and transition metals. Phyllosilicates, namely silicates having a structure in which the SiO₄ tetrahedra are arranged in layers between which are enclosed the metal cations, are preferably used. A family of silicates which is particularly preferred as tightening agents is that of the laponites. The laponites are sodium magnesium lithium silicates having a layered structure similar to that of montmorillonites. Laponite is the synthetic form of the natural mineral known as “hectorite”. Use may be made, for example, of the laponite sold under the name Laponite XLS or Laponite XLG by Rockwood.

Yet another class of tightening agents is composed of wax microparticles. They are particles having a diameter generally of less than 5 μm or better still of less than 0.5 μm and composed essentially of a wax or of a mixture of waxes chosen, for example, from carnauba wax, candelilla wax or esparto wax. The melting point of the wax or of the mixture of waxes is preferably between 50° C. and 150° C.

In another alternative form, use may be made, as tightening agent, of colloidal particles of inorganic fillers. The term “colloidal particles” is understood to mean colloidal particles, in dispersion in an aqueous, aqueous/alcoholic or alcoholic medium, having a number-average diameter of between 0.1 and 100 nm, preferably between 3 and 30 nm. Mention may be made, as examples of inorganic fillers, of silica, cerium oxide, zirconium oxide, alumina, calcium carbonate, barium sulphate, calcium sulphate, zinc oxide and titanium dioxide. An inorganic filler which is particularly preferred is silica. Colloidal silica particles are available in particular in the form of an aqueous colloidal silica dispersion from Catalysts & Chemicals under the trade names Cosmo S-40 and Cosmo S-50. Use may also be made of composite silica/alumina colloidal particles, such as those sold by Grace under the names of Ludox AM, Ludox HSA and Ludox TMA.

XIII) Mattifying fillers, the term “mattifying filler” denoting a porous or nonporous and spherical or non-spherical particle exhibiting a refractive index of less than or equal to 2.2, in particular of less than or equal to 2 and especially of less than or equal to 1.8, preferably ranging from 1.3 to 1.6. The mattifying fillers according to the invention generally have a size by volume of less than 15 μm.

In a preferred form of the invention, the mattifying fillers are spherical.

In a preferred form of the invention, the mattifying fillers are porous. In this case, the specific surface of the particles, which can be linked to the porosity, is greater than 10 m²/g, preferably greater than 50 m²/g.

More particularly, these fillers can, for example, be chosen from:
porous silica microparticles, such as, for example, Silica beads SB150 and SB700 from Miyoshi having a mean size of 5 microns; Sunspheres Series-H from Asahi Glass, such as, for example, Sunsphere H33, H51 and H53 with respective sizes of 3, 5 and 5 microns;
polytetrafluoroethylene powders, such as the Ceridust 9205F PTFEs from Clariant having a mean size of 8 microns;
silicone resin powders, such as the Tospearl 145A silicone resin from GE Silicone having a mean size of 4.5 microns;
hollow hemispherical silicone particles, such as the NLK 500, NLK 506 and NLK 510 products from Takemoto Oil and Fat;
powders formed of acrylic copolymers, in particular of polymethyl (meth)acrylate, such as the Jurymer MBI PMMA particles from Nihon Junyoki having a mean size of 8 microns, the hollow PMMA spheres sold under the name Covabead LH85 by Wackherr, the polymethyl methacrylate microspheres sold under the name Microsphere M-100 by Matsumoto or the microspheres sold under the name Micropearl F 80 ED by Matsumoto;
wax powders, such as the “Paraffin Wax Microease 114S” particles from Micropowders having a mean size of 7 microns;
polyethylene powders, in particular comprising at least one ethylene/acrylic acid copolymer and especially composed of ethylene/acrylic acid copolymers, such as the Flobeads EA 209 particles from Sumitomo (with a mean size of 10 microns);
crosslinked organopolysiloxane elastomer powders which are coated with silicone resin, in particular with silsesquioxane resin, such as described, for example, in the document U.S. Pat. No. 5,538,793. Such elastomer powders are sold under the names “KSP-100”, “KSP-101”, “KSP-102”, “KSP-103”, “KSP-104” and “KSP-105” by Shin-Etsu;
composite talc/titanium dioxide/alumina/silica powders, such as those sold under the name Coverleaf AR-80 by Catalysts & Chemicals;
polyamide (Nylon™) powders, such as, for example, the Nylon 12 particles of orgasol type from Atofina having a mean size of 10 microns;
microspheres based on acrylic copolymers, such as those of ethylene glycol dimethacrylate/lauryl methacrylate copolymer sold by Dow Corning under the name of Polytrap;
the expanded powders, such as hollow microspheres and in particular the microspheres formed of a terpolymer of vinylidene chloride, of acrylonitrile and of methacrylate sold under the name Expancel by Kemanord Plast under the references 551 DE 12 (particle size of approximately 12 μm and density of 40 kg/m³), 551 DE 20 (particle size of approximately 30 μm and density of 65 kg/m³) and 551 DE 50 (particle size of approximately 40 μm);
ethylene/acrylate copolymer powders, such as those sold under the name Flobeads by Sumitomo Seika Chemicals;
powders formed of natural organic materials, such as powders formed of starch, in particular of maize, wheat or rice starches, which may or may not be crosslinked, such as the powders formed of starch crosslinked by octenylsuccinic anhydride sold under the name Dry-Flo by National Starch;
microbeads formed of cellulose and of fibres;
and their mixtures.

The composition according to the invention can additionally include various additional fillers of inorganic or organic origin. They can be of any shape, in particular platelet, spherical or oblong, whatever their crystallographic form (for example, leaf, cubic, hexagonal, orthorhombic, and the like).

Mention may in particular be made, among the additional fillers which can be used in the composition according to the invention, of talc, mica, kaolin, of poly-β-alanine and of polyethylene, lauroyllysine, starch, boron nitride, precipitated calcium carbonate, magnesium carbonate, basic magnesium carbonate, barium sulphate, hydroxyapatite, glass or ceramic micro-capsules and metal soaps derived from organic carboxylic acids having from 8 to 22 carbon atoms, in particular from 12 to 18 carbon atoms, for example zinc stearate, magnesium stearate, lithium stearate, zinc laurate or magnesium myristate.

XIV) Hair treatment active principles, such as (1) agents which inhibit hair loss and agents which stimulate hair growth, such as minoxidil, biotin, aminexil, cysteine, finasteride, 2,4-dipyrimidine N-oxide, panthenol and derivatives, flavanone T, calcium antagonists, such as diltiazem, verapamil, alverine and nifedipine, hormones, such as progesterone, FP receptor agonists, such as latanoprost, type 1 15-hydroxyprostaglandin dehydrogenase inhibitors, prostaglandins and their derivatives, or more generally any plant extract having an activity against 5α-reductase of type I or II; (2) antidandruff agents, such as zinc pyrithione, 1-hydroxy-2-pyrrolidone derivatives or selenium sulphides.

The aqueous composition and/or the film can also comprise the adjuvants commonly used in the cosmetic or dermatological field, such as dyes, pigments, fragrances and essential oils, electrolytes, cosmetic active principles, pH adjusters (acid or base) or preservatives.

The aqueous composition can also comprise polymers which can be of use in contributing specific rheological properties to the aqueous composition. Mention may be made, as polymers, for example, of modified or unmodified carboxyvinyl polymers, such as the products sold under the names Carbopol (INCI name: carbomer) and Pemulen (INCI name: Acrylates/C10-30 alkyl acrylate crosspolymer) by Noveon; polyacrylates and polymethacrylates, such as the products sold under the names of Lubrajel and Norgel by Guardian or under the name Hispagel by Hispano Chimica; polyacrylamides; optionally crosslinked and/or neutralized polymers and copolymers of 2-acrylamido-2-methylpropanesulphonic acid, such as the poly(2-acrylamido-2-methylpropanesulphonic acid) sold by Clariant under the name “Hostacerin AMPS” (INCI name: ammonium polyacryldimethyltauramide); crosslinked anionic copolymers of acrylamide and of AMPS which are provided in the form of a W/O emulsion, such as those sold under the name of Sepigel 305 (INCI name: Polyacrylamide/C13-14 isoparaffin/laureth-7) and under the name of Simulgel 600 (INCI name: Acrylamide/sodium acryloyldimethyltaurate copolymer/isohexadecane/polysorbate 80) by Seppic; polysaccharide biopolymers, such as xanthan gum, guar gum, locust bean gum, gum acacia, scleroglucans, derivatives of chitin and of chitosan, carrageenans, gellans, alginates, celluloses, such as microcrystalline cellulose, carboxymethylcellulose, hydroxymethylcellulose and hydroxypropylcellulose; maleic anhydride copolymers, such as the methyl vinyl ether/maleic anhydride copolymer crosslinked by 1,9-decadiene (INCI name: PVM/MA decadiene crosspolymer) sold under the name Stabileze OM or 06 by ISO; associative polymers, such as associative polyurethanes, copolymers comprising at least two lipophilic hydrocarbon chains having 6 to 30 carbon atoms separated by a hydrophilic block, such as the polyurethanes sold under the names Serad FX1010, Serad FX1100 and Serad FX1035 by Hüls (INCI name: Polyurethane), those sold under the names Rheolate 255, Rheolate 278 and Rheolate 244 by Rheox (INCI name: Polyether-urea-polyurethane), those sold under the names DW 1206F, DW 1206J, DW 1206B and DW 1206G by Röhm & Haas (INCI name: Polyurethane) and that sold under the name Acrysol RM 2020 by Röhm & Haas. Use may also be made of fixing polymers, such as those used conventionally in lacquers and styling products for the hair.

The adjuvants mentioned above can be present in the final product in the amounts usual in the field under consideration, these amounts depending on the compound used and the purpose desired. The adjuvants can be present, for example, in an amount ranging from 0.001 to 20% by weight and better still from 0.01 to 10% by weight, with respect to the weight of the final product.

The kit according to the invention can be used in particular to produce products intended to be applied to the skin, mucous membranes, superficial body growths or hair, in particular as care products for the skin or as makeup products for the skin or as hair products or as antisun products.

The examples below of compositions according to the invention are given by way of illustration and without a limiting nature. The amounts are given therein as % by weight, unless otherwise mentioned, and the names are shown as chemical name or INCI name, as the case may be.

### Example 1 and Comparative Example

### Anhydrous Film

| | | Example 1 |
|---|---|---|
| | Comparative | according to |
| | example | the invention |
| Composition | Amount in % | Amount in % |
| Polyvinyl alcohol (Celvol | 2.1 | 2.0 |
| 540 PVA) | | |
| Hydroxypropylcellulose | 3.2 | 3.1 |
| (Klucel EF) | | |
| Water | 87.0 | 85.3 |
| Carrageenan | 1.0 | 1.0 |
| Esterified maize starch | 1.6 | 1.6 |
| (Dry Flo Plus) | | |
| Glycerol | 3.2 | 3.1 |
| Polyethylene glycol 400 | 1.9 | 1.9 |
| PEG-12 dimethicone | — | 2.0 |
| (Silsoft 880) | | |

Procedure for the Preparation of the Film

The procedure comprised two stages. In a first step, the ingredients were mixed and, in a second step, this mixture was spread and then dried in order to form the film.

1) Preparation of the Solution

The constituents shown in the table above were mixed in two stages as described below and then the mixture was homogenized using a turbine mixer (Rayneri).

The procedure for the preparation of the solution was carried out in two stages in the following way:

1^{st} stage: the polymers (polyvinyl alcohol and hydroxypropylcellulose) were dispersed in water and stirring was carried out at the speed and at the time necessary in order to obtain a homogeneous gel. This stage lasted three hours with vigorous stirring (1200 to 1600 rpm).

2^{nd} stage: the carrageenan and, for the example according to the invention, the oxyethylenated polysiloxane were dispersed and mixing was carried out until a perfect homogeneous mixture was obtained. Once the mixture was homogenized, the starch, the PEG 400 and the glycerol were added while stirring at the same speed as above. This stage lasted 20 minutes.

2) Formation of the Film

The coating of the mixture was carried out using a film drawer on a sheet of Silphan S100 M44A on the non-silicone face. The equipment used was an automatic film applicator from Braive Instruments. The wet thickness chosen was 700 μm. The films were subsequently placed in an oven at 55° C. without humidity control for 24 hours.

After drying, anhydrous films with a thickness of approximately 50 μm were obtained, the compositions of which were as follows:

| | | Example 1 |
|---|---|---|
| | Comparative | according to |
| | example | the invention |
| Composition | Amount in % | Amount in % |
| Polyvinyl alcohol (Celvol | 16.2 | 13.6 |
| 540 PVA) | | |
| Hydroxypropylcellulose | 24.6 | 21.1 |
| (Klucel EF) | | |
| Carrageenan | 7.7 | 6.8 |
| Esterified maize starch | 12.3 | 10.9 |
| (Dry Flo Plus) | | |
| Glycerol | 24.6 | 21.1 |
| Polyethylene glycol 400 | 14.6 | 12.9 |
| PEG-12 dimethicone | — | 13.6 |
| (Silsoft 880) | | |

Test

A dissolution test was carried out in order to determine the dissolution properties of the film of Example 1 in comparison with that of the comparative example.

Description of the Method:

The method used consists in measuring the time which a fluid formulation (hereinafter referred to as liquor) has to take to pass through a water-soluble film, using in order to do this a Franz cell diverted from its normal use.

The film was placed, using a pair of pincers, between the top part and the bottom part of a Franz cell. A known amount of the liquor was deposited on the film using a Finn pipette (20-200 μl) or a Microman pipette (0-50 μl), without contact occurring between the tip of the pipette and the film.

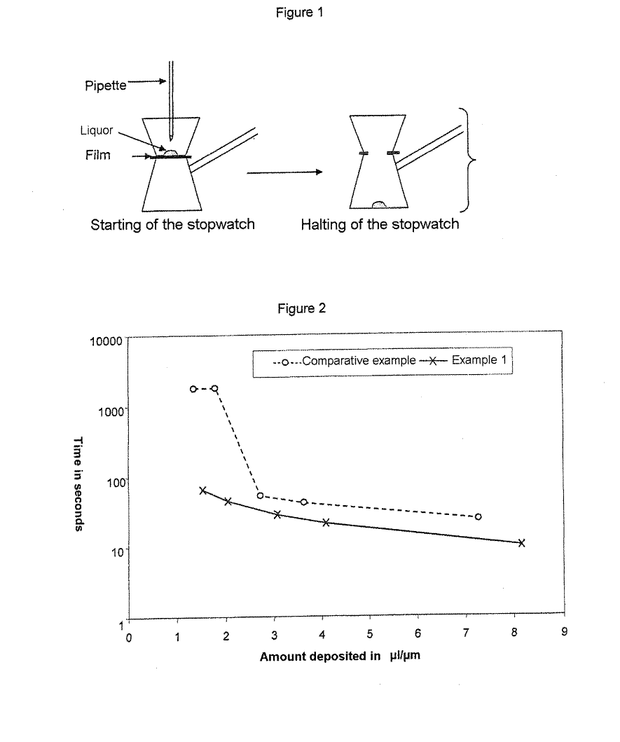

The test arrangement is represented in FIG. 1.

The stopwatch was started when the drop of liquor was deposited on the film. The stopwatch was subsequently halted when the drop deposited had passed through the film and had touched the bottom of the Franz cell.

The time necessary for amounts of liquor varying from 50 to 400 μl to pass through the film was thus measured. These values were subsequently used to construct a curve representing the time for passing through the film (in seconds) as a function of the amount of liquor deposited, standardized by the thickness of the film, i.e. 50 μm (in μl/μm). The curves obtained with the films corresponding to the examples according to the invention (Example 1) and the comparative example are given in FIG. 2.

It is found that the film of Example 1 of the invention, which comprises 13% (in the dry form) of an oxyethylenated polysiloxane (Silsoft 880), exhibited dissolution kinetics which are very markedly improved with respect to the comparative example.

Example of the aqueous composition in which the film can be dissolved

Aqueous composition (serum)
Phase A

| | |
|---|---|
| Xanthan gum | 0.2% |
| PVM/MA decadiene crosspolymer | 0.2% |
| Methylparaben | 0.2% |
| Phenoxyethanol | 0.35% |
| Water | q.s. for 100% |

Phase B

| | |
|---|---|
| Triethanolamine | 0.2% |
| Polyacrylamide/C13-C14 | 1% |
| isoparaffin/laureth-7 (Sepigel 305) | |
| Diazolidinylurea | 0.3% |
| Glycerol | 7% |

Procedure for the preparation of the serum: Phase A was heated to 75° C. approximately with stirring and then phase B, prepared beforehand, was poured into phase A. Subsequently, heating was halted while continuing to stir until the mixture had returned to ambient temperature. Gentle stirring was subsequently maintained for 30 minutes.

The aqueous composition (serum) and one or more films of Example 1 can be presented in the form of a kit. During use, the consumer mixes, in the hollow of her hand using the fingers, one or more films of Example 1 with a dose of between 100 and 500 mg of the aqueous composition for approximately 10 seconds. She gently massages the surface to be treated so as to promote the spreading over the skin of the product thus obtained.

The final product obtained can be applied to the face or the body, in particular to the face, for all types of cosmetic treatments depending on the active principles added to the composition, for example as antiageing composition, as mattifying composition, in particular for greasy skin, for the depigmentation of the skin, as slimming composition, for the photoprotection of the skin, for correcting dyschromia, to give a good appearance effect, as selftanning product, and the like.

The above written description of the invention provides a manner and process of making and using it such that any person skilled in this art is enabled to make and use the same, this enablement being provided in particular for the subject matter of the appended claims, which make up a part of the original description and including a water-soluble anhydrous film comprising (i) at least one water-soluble or water-dispersible film-forming polymer, (ii) at least one polysaccharide thickening agent, (iii) at least one water-soluble or water-dispersible oxyalkylenated polysiloxane and (iv) at least one plasticizer chosen from polyols.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A water-soluble anhydrous film comprising (i) at least one water-soluble or water-dispersible film-forming polymer, (ii) at least one polysaccharide thickening agent, (iii) at least one water-soluble or water-dispersible oxyalkylenated polysiloxane and (iv) at least one plasticizer chosen from polyols.

2. The film according to claim 1, wherein said film has a thickness of 10 μm to 1000 μm.

3. The film according to claim 1, wherein said film comprises 0 to 15′ of water.

4. The film according to claim 1, comprising at least one oxyalkylenated polysiloxane that has a solubility in water, measured at 25° C., of at least 0.1 gram/litre.

5. The film according to claim 1, comprising at least one oxyalkylenated polysiloxane chosen from the water-soluble silicones of formula (a):
R²₃SiO(R²₂SiO)ₚ(R²PESiO)_{q}SiR²₃ (a)
in which:
the R² radicals, which are identical or different, denote a monovalent hydrocarbon radical chosen from alkyl, aryl and aralkyl radicals having at most 10 carbon atoms;
p varies from 0 to 150;
q varies from 1 to 12;
the polyether group PE has the following formula (b):
—CₓH₂ₓ(OC₂H₄)_{y}(OC₃H₆)_{z}OR³ (b)
in which:
x varies from 1 to 8;
y is greater than 0;
z is greater than or equal to 0; the values of y and z being such that the total molecular weight of the polyoxyalkylene portion of the polyether group PE varies from 200 to 10 000;
R³ denotes hydrogen, a C₁-C₈ alkyl group or a C₂-C₈ acyl group.

6. The film according to claim 5, wherein in formula (a) the R² radicals are chosen from C₁-C₄ alkyl groups, hexyl, phenyl and benzyl groups.

7. The film according to claim 5, wherein in formula (a) the R³ radicals are chosen from C₁-C₄ alkyl groups.

8. The film according to claim 1, comprising at least one oxyalkylenated polysiloxane chosen from the water-soluble silicones of formula (a′):
Me₃SiO(MeSiO)ₚ(MePESiO)_{q}SiMe₃ (a′)
in which:
p varies from 0 to 150;
q varies from 1 to 12;
Me denotes the methyl radical;
PE denotes:
—(CH₂)₃(OC₂H₄)_{y}(OC₃H₆)_{z}OR³ (b′)
in which:
y is greater than 0;
z is greater than or equal to 0; the values of y and z being such that the total molecular weight of the polyoxyalkylene portion of the polyether group PE varies from 200 to 10 000;
R³ denotes hydrogen or a C₁-C₄ alkyl group.

9. The film according to claim 1, comprising at least one oxyalkylenated polysiloxane chosen from branched silicones of following formula (c):
(MeSiO)_{q-2}[SiOMe₂)_{p/q}OPE]_{q} (c)
in which:
p varies from 0 to 150;
q varies from 1 to 12;
Me denotes the methyl radical;
PE denotes the group of following formula (d):
—(OC₂H₄)_{y}(OC₃H₆)_{z}R³ (d)
in which:
y is greater than 0;
z is greater than or equal to 0; the values of y and z being such that the total molecular weight of the polyoxyalkylene portion of the polyether group PE varies from 200 to 10 000;
R³ denotes a C₁-C₄ alkyl group.

10. The film according to claim 1, wherein the amount of oxyalkylenated polysiloxane(s) ranges from 0.5 to 30% by weight with respect to the total weight of the film.

11. The film according to claim 1, wherein the film-forming polymer has a solubility in water, measured at 25° C., at least equal to 0.1 gram/litre.

12. The film according to claim 1, wherein the film-forming polymer is chosen from vinyl polymers, cellulose derivatives, starches and their derivatives, optionally modified polymers of natural origin, polymers derived from chitin or from chitosan, protein polymers, acrylic copolymers of phosphorylcholine, anion-cation complexes, and their mixtures.

13. The film according to claim 1, wherein the film-forming polymer is chosen from vinyl polymers, cellulose derivatives and their mixtures.

14. The film according to claim 1, wherein the film-forming polymer is chosen from polyvinyl acetate, hydroxypropylcellulose, hydroxypropylmethylcellulose and their mixtures.

15. The film according to claim 1, wherein the amount of water-soluble film-forming polymer(s) ranges from 10 to 95% by weight with respect to the total weight of the film.

16. The film according to claim 1, wherein the polysaccharide thickening agent is chosen from gum arabic, gum ghatti, gum karaya, locust bean gum, guar gum, tamarin gum, xanthan gum, gellan, pectins, gum tragacanth, agar-agar, alginates, carrageenan, furcelleran, konjac gum, cellulose derivatives, and their mixtures.

17. The film according to claim 1, wherein the polysaccharide thickening agent is chosen from carrageenans.

18. The film according to claim 1, wherein the amount of thickening agent(s) ranges from 0.5 to 40% by weight with respect to the total weight of the film.

19. The film according to claim 1, wherein the amount of polyol plasticizer(s) ranges from 1 to 40% by weight with respect to the total weight of the film.

20. A product obtained by mixing at least one water-soluble anhydrous film according to claim 1 with an aqueous composition.

21. The product according to claim 20, wherein the aqueous composition is a solution or emulsion.

22. The product according to claim 20, wherein the thin film or films and/or the aqueous composition further comprise one or more active principles.

23. Kit for the formulation of a cosmetic product comprising (1) an aqueous composition and (2) at least one water-soluble anhydrous film according to claim 1.

24. The kit according to claim 23, comprising a plurality of identical and/or different water-soluble anhydrous films.
